(19) United States
(12) Patent Application Publication
(10) Pub. No.: US 2008/0207500 A1
(43) Pub. Date: Aug. 28, 2008

(54) Medicament

(76) Inventor: Deirdre McIntosh, East Sussex (GB)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 11/631,067

(22) PCT Filed: Jul. 8, 2005
(86) PCT No.: PCT/GB2005/050108

§ 371 Date: Dec. 7, 2007

(30) Foreign Application Priority Data
Jul. 8, 2004 (GB) 0415359.9
Oct. 21, 2004 (GB) 0423386.2

(51) Int. Cl. A61K 38/22; C07K 14/575; C07K 1/00; A61P 25/00

(52) U.S. Cl. 514/12; 530/324; 530/344

(57) ABSTRACT

Analysis of a goat serum product with many therapeutic effects is described. The product is identified as containing proopiomelanocortin (POMC) and Corticotropin releasing factor (CRF) peptides, as well as breakdown products of these peptides. We describe methods of treatment of diseases including cancers, multiple sclerosis, and neural disorders using these peptides and their products, as well as medicaments including such peptides and methods of producing the peptides.

### FIELD OF THE INVENTION

The present invention relates to a medicament, and in particular to a pharmaceutical composition. The medicament is considered particularly suited to treatment of neural disorders, although a number of other disorders may be treatable with the invention. Aspects of the invention also relate to methods of preparation of such a medicament, and to methods of treatment of disorders using said medicament.

### BACKGROUND TO THE INVENTION

PCT publications WO03/004049 and WO03/064472 describe therapeutic agents and treatments which are based on a serum composition with many surprising beneficial effects. The respective content of each of these two texts is incorporated in full by specific reference. In particular, the reader is referred to them for an understanding of how the therapeutic agent can be prepared, and for the indications which can be treated.

Typically a goat is immunised with HIV-3B viral lysate raised in H9 cells. The resulting serum is believed to be active against, among other disorders, multiple sclerosis. The reader is further referred in particular to the section on pages 3 and 4 of WO03/004049 headed ‘Example of Production of Goat Serum’ for further details of the production of serum. This section is incorporated herein by reference. A brief summary is given below.

### Preparation of Serum

Approximately 400 cc of blood is taken from a goat under sterile technique. The animal may typically be re-bled in 10 to 14 days, once the volume of blood is replenished A pre-bleeding regime may be useful to stimulate production of the active components of the serum. The blood is then centrifuged to separate the serum, and the serum filtered to remove large clots and particulate matter. The serum is then treated with supersaturated ammonium sulphate (47% solution at 4° C.) to precipitate antibodies and other material. The resulting solution is centrifuged in a Beckman J6M/E centrifuge at 3500 rpm for 45 minutes, after which the supernatant fluid is removed. The precipitated immunoglobulin and other solid material are resuspended in PBS buffer (phosphate buffered saline) sufficient to redissolve the precipitate.

The solution is then subjected to diafiltration against a PBS buffer with a molecular weight cut-off of 10,000 Daltons. at 4° C. After diafiltration the product is filtered through a 0.2 micron filter into a sterile container and adjusted to a protein concentration of 4 mg/ml. The solution is put into vials to give single doses of 1 ml, and stored at −22° C. prior to use. This product is referred to herein as the serum composition, the composition, or the product, while treatment of a patient involves administering the composition to the patient by an appropriate route (usually subcutaneously).

The use of HIV-3B viral lysate as an immunogen is not believed to be essential for the production of active serum; it is believed that a medium which has been used for growth of a viral culture, or which is suitable for such growth, may also produce a suitable response when used as an immunogen. The supernate of a cell culture growth medium such as PBMC or the cancer immortal cell line as used to grow HIV-3B are given as an example. The HIV or other virus does not need to be present to produce an effective immunogen to create the composition. Other suitable immunogens are recited on pages 12 and 13 of WO03/064472.

A pyrogenic material (for example, RIBI or Freund's adjuvant) can be used to promote production of the active component in the serum. Another possible factor may be exposure of the animal to daylight, with greater daylight hours (or exposure to daylight equivalent) may increase active component serum levels.

### Uses of the Serum Composition

The composition is believed to be effective against a number of disorders, in particular multiple sclerosis. Reference is also made in the previously-identified publications to the composition as being useful in the treatment of inflammatory diseases such as rheumatoid arthritis; optic neuritis; motor neurone disease; autoimmune diseases; axonal or nerve damage; and cancers. The composition is also believed to cause a reduction in viral load in HIV patients, and an increase in CD4+ cells.

Several other diseases which may be treated by the composition are described, and the reader is referred to these earlier publications for a full understanding of the range and nature of conditions which may be treated. In particular, the contents of WO03/004049 and WO03/064472 are specifically incorporated herein by reference.

A non-exhaustive list of disorders against which the serum composition is believed to be effective, in addition to those mentioned above, includes cancers, in particular myelomas, melanomas, and lymphomas; cardiovascular diseases; and neural disorders, both demyelinating and non-demyelinating.

Examples of disorders which may be treated in accordance with the present invention include cerebrovascular ischaemic disease; Alzheimer's disease; Huntingdon's chorea; mixed connective tissue diseases; scieroderma; anaphylaxis; septic shock; carditis and endocarditis; wound healing; contact dermatitis; occupational lung diseases; glomerulnephritis; transplant rejection; temporal arteritis; vasculitic diseases; hepatitis; and burns. All of these disorders may have an inflammatory component, but are believed to be additionally treatable based on the non-demyelinating neural aspect of the disorder. Further non-demyelinating disorders which may be treated, and which are considered to have a degenerative component include multiple system atrophy; epilepsy; muscular dystrophy; schizophrenia; bipolar disorder; and depression. Other non-demyelinating disorders which may be treated include channelopathies; myaesthenia gravis; pain due to malignant neoplasia; chronic fatigue syndrome; fibromyositis; irritable bowel syndrome; work related upper limb disorder; cluster headache; migraine; and chronic daily headache.

Demyelinating disorders which may be treatable include infections of the nervous system; nerve entrapment and focal injury; traumatic spinal cord injury; brachial plexopathy (idiopathic and traumatic, brachial neuritis, parsonage turner syndrome, neuralgic amyotrophy); radiculopathy; channelopathies; and tic douloureux.

The composition may be useful in the treatment of autoimmune diseases including lupus, psoriasis, eczema, thyroiditis, and polymyositis.

The composition is also believed to be effective against inflammatory conditions.

The composition is useful in the treatment of all kinds of peripheral neuropathy of axonal and demyelinating type, including hereditary motor and sensor neuropathy of all types; Charcot-Marie-Tooth disease (CMT) types CMT1A, CMT1B, CMT2, CMT3 (Dejerine Sottas disease), CMT4 (Types A, B C and D), X-linked Charcot-Marie-Tooth disease (CMTX); Hereditary Neuropathy with liability to pressure palsies (HNPP)—also called Tomaculous neuropathy; Hereditary Motor and Sensory Neuropathy with Deafness—Lom (HMSNL); Proximal Hereditary Motor and Sensory Neuropathy/Neuronopathy (HMSNP); Hereditary Neuralgic Amyotrophy; Hereditary Sensory and Autonomic Neuropathies (HSAN1, HSAN2, HSAN3 (also called Riley-Day syndrome or familial dysautonomia), HSAN4, HSAN5); Familial Amyloid polyneuropathies (Type I, Type II, Type III, Type IV); Metachromatic Leukodystrophy; Krabbe's Disease; Fabry's Disease; Adrenoleukodystrophy; Refsum's disease (HMSN IV); Tangier Disease; Friedreich's ataxia; Spinal cerebellar ataxia (SCA) all types—SCA1, SCA2, SCA3, SCA4, SCA5, SCA6, SCA7, SCA8, SCA10, SCA11, SCA12, SCA13, SCA14, SCA16; Spinocerebellar Ataxia; Cockayne's syndrome; and Giant axonal neuropathy.

The composition may also be useful in the treatment of chronic inflammatory demyelinating polyneuropathy (CIDP), and Guillain-Barre syndrome.

The composition may also have anti-angiogenic properties, caused by the molecules thrombospondin-1 (TSP-1) and platelet factor-4 (PF-4).

It is believed that the composition may also be effective for treatment of animals, in particular, but not exclusively, the treatment of canine atopic dermatitis, canine oral melanoma, and equine pulmonary disorders.

### Nature of the Serum

Although the serum composition has exhibited many surprising effects, and has been studied extensively, until now the active component or components of the serum have not yet been identified. This has been disadvantageous, both in terms of isolating the active component for further study, and in terms of exploring possible alternative sources of the active component or components. Further, it has been necessary to administer the treatment to patients as serum, which necessitates injections, and imposes certain restrictions on the handling and processing of the composition. It is believed that the serum has bioactive components sensitive to protease degradation.

We have now identified a number of potential active components of the serum. This identification allows the manufacture of novel pharmaceutical compositions comprising one or more of the active components in various forms, and the treatment of one or more of the disorders recited above and in our earlier patent applications using the active component(s). The identification also opens up novel approaches for treating the various disorders based on the active component(s) and not simply the serum itself.

### SUMMARY OF THE INVENTION

The invention resides in a bioactive composition which triggers a molecular cascade in treated patients.

According to a first aspect of the present invention, there is provided a pharmaceutical composition comprising a corticotropin releasing factor (CRF) peptide. CRF is also known as corticotropin releasing hormone (CRH).

CRF is a peptide produced in the hypothalamus, and is believed to be involved in stress response. Human CRF is described in detail in entry 122560 of OMIM (online mendelian inheritance in man, accessible through http://www.ncbi.nlm.nih.gov/). The nucleotide and amino acid sequence of human CRF is also known, and has GENBANK accession number BC011031. Knowledge of the sequence and size data for human CRF will allow the skilled person to determine the equivalent information for non-human CRF, including goat CRF.

By “a CRF peptide” is meant any peptide having a corresponding sequence, structure, or function. It will be apparent to the skilled person that the canonical nucleotide and/or amino acid sequences given for human CRF in the GENBANK entry referenced above may be varied to a certain degree without affecting the structure or function of the peptide. In particular, allelic variants and functional mutants are included within this definition. Mutants may include conservative amino acid substitutions; and fragments and derivatives of CRF.

Administration of CRF to a patient is believed to stimulate production of endogenous CRF, which in turn stimulates production of proopiomelanocortin (POMC) and its related component peptides.

POMC is a peptide (prohormone) produced in the pituitary gland (as well as a number of other organs, certain tumours such as melanomas, and normal skin cells) which is the precursor of a set of corticotrophic hormones which exert a number of effects on the host. POMC is the precursor to alpha, beta, and gamma melanocyte stimulating hormone (MSH); adrenocorticotrophin (ACTH); beta and gamma lipotropin (LPH); and beta endorphin. All of these hormones are cleaved from a single large precursor, POMC, and are termed herein “POMC products”.

Preferably the pharmaceutical composition comprises non-human CRF; conveniently ungulate CRF; and most preferably goat CRF. It has been surprisingly identified that goat serum contains CRF, particularly when the goat is stimulated by physiological stress, such as bleeding or immunization. This provides a convenient source for CRF for pharmaceutical compositions of the present invention. It is also believed that CRF may have a self-sustaining effect in the patient, in that administration of an initial amount of CRF leads to endogenous production of CRF in the patient; thus, an initial administration of a low level of CRF may have a significant effect on the patient, including an increase in the levels of POMC peptides.

Administration of pharmaceutical compositions of the invention may be accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, such compositions may comprise suitable pharmaceutically acceptable carriers comprising excipients and other components which facilitate processing of the active compounds into preparations suitable for pharmaceutical administration.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical preparations for oral use can be obtained through combination of active compounds with a solid excipient, optionally grinding a resulting mixture, and processing the mixture of granules, after adding suitable additional compounds if desired to obtain tablets or dragee cores. Suitable excipients include carbohydrate or protein fillers such as sugars, including lactose, sucrose, mannitol, sorbitol; starch from corn, wheat, rice, potato, or other plants; cellulose such as methylcellulose, hydroxypropylmethylcellulose, or sodium carboxymethylcellulose; and gums including arabic and tragacanth; as well as proteins such as gelatin and collagen. If desired, disintegrating or solubilising agents may be added, such as cross linked polyvinyl pyrrolidone, agar, alginic acid, or a salt thereof.

Dragee cores can be provided with suitable coatings such as concentrated sugar solutions, which may also contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for product identification or to characterise the quantity of active compound.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally stabilisers. In soft capsules, the active compounds can be dissolved or suspended in suitable liquids, such as fatty oils, liquid paraffin, or liquid polyethylene glycol with or without stabilisers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions.

For topical or nasal administration, penetrants appropriate to the particular barrier to be permeated may be used in the formulation.

The pharmaceutical compositions of the present invention can be manufactured substantially in accordance with standard manufacturing procedures known in the art.

The composition may also comprise one or more peptide regulatory or releasing factors, which may induce a cascade of release of further peptides by a variety of cells in the patient. Such additional factors are preferably derived from the same source as the CRF, in particular goat serum. Suitable factors include α-HLA, TGF-β, and IL-10, among others.

In preferred embodiments, the composition may comprise one or more of vasopressin, beta endorphin, and an enkephalin. In certain embodiments, the composition may comprise CRF binding protein, CRF-BP. This binds CRF and may act as a reservoir for subsequent release of CRF to the patient.

The composition may further comprise a POMC peptide or a POMC product; certain POMC products may be useful to administer to a patient to stimulate further production, or to obtain a desired response before endogenous POMC can be produced.

Human POMC is described in detail in entry 176830 of OMIM (online mendelian inheritance in man, accessible through http://www.ncbi.nlm.nih.gov/). The nucleotide and amino acid sequence of human POMC is also known, and has GENBANK accession number BC065832. Human POMC gives rise to a glycosylated protein precursor having a molecular weight of 31 kDa.

By “a POMC peptide” is meant any peptide having a corresponding sequence, structure, or function. It will be apparent to the skilled person that the canonical nucleotide and/or amino acid sequences given for human POMC in the GENBANK entry referenced above may be varied to a certain degree without affecting the structure or function of the peptide. In particular, allelic variants and functional mutants are included within this definition. Mutants may include conservative amino acid substitutions. “A POMC peptide” as used herein refers to any peptide acting as a precursor to at least one form of MSH, ACTH, at least one form of LPH, β endorphin, met-enkephalin and leu-enkephalin; and preferably all of α, β, and γ MSH; ACTH; β and γ LPH; and β endorphin, met-enkephalin and leu-enkephalin

Although research has been carried out into the pharmaceutical potential of certain of the individual products of the POMC peptide, it is believed that administration of POMC itself has not been determined to have any medical use. It is possible that POMC itself is inactive, and must be cleaved into its products before having an activity.

Preferably the pharmaceutical composition comprises non-human POMC; conveniently ungulate POMC; and most preferably goat POMC. Although POMC is produced in the pituitary gland, and so would not be expected to be present in serum, at least at significant levels, it has been surprisingly identified that goat serum contains POMC, POMC-related peptides, and molecules associated with the POMC cascade, particularly when the goat is stimulated by physiological stress, such as bleeding or immunization. This provides a convenient source for POMC for pharmaceutical compositions of the present invention. It is also believed that POMC may have a self-sustaining effect in the patient, in that administration of an initial amount of POMC leads to endogenous production of POMC in the patient; thus, an initial administration of a low level of POMC may have a significant effect on the patient.

It is believed that, on administration of POMC and its associated molecules to a subject, the peptide is proteolysed to provide one or more of the products of POMC in a readily available form to the subject; there is also the induction of a molecular cascade which stimulates the hypothalamo-pituitary-adrenal axis (HPA). This would be consistent with previously observed effects of the unpurified goat serum; for example, a rapid ‘buzz’ effect on sublingual administration may be due to proteolysis of POMC to release β endorphin, which can then be absorbed through the mucous membranes. In addition, alpha MSH is known to have an effect on IL-10 and TGF-β production, which results in an anti-inflammatory effect, consistent with that which has been observed with goat serum. Alpha MSH is also known to inhibit the release of pro-inflammatory cytokines.

The composition may also have anti-inflammatory properties. We believe there is a passive transfer of anti-inflammatory response from the goat or other animal used to produce the serum to the patient. This is a consequence of the purification process used to prepare the composition, in which a variety of active factors are retained in the serum. The composition may also comprise additional active components which provide an anti-inflammatory effect.

As mentioned above, it is believed that an initial administration of POMC (optionally together with CRF and/or vasopressin) may stimulate native production of POMC and its regulatory peptides. A further aspect of the present invention therefore provides a method of stimulating POMC production in a patient, comprising administering exogenous POMC to the patient. The exogenous POMC is preferably non-human, and more preferably goat POMC. Conveniently, administration is subcutaneous; this gives a subcutaneous depot of active composition for subsequent slow release into the patient's system.

A further aspect of the present invention provides a pharmaceutical composition comprising a POMC peptide.

According to a further aspect of the invention, there is provided a pharmaceutical composition comprising two or more of alpha, beta, and gamma melanocyte stimulating hormone (MSH); adrenocorticotrophin (ACTH); beta and gamma lipotropin (LPH); and beta endorphin. Given the likely proteolysis of POMC on administration, it may be possible to achieve similar effects by administration of two or more of the individual hormones derived from POMC. The pharmaceutical composition may provide the recited hormones as individual peptides, or as one or more precursor molecules (for example, partial breakdown products of POMC). Preferably three, four, five, six, or seven of the hormones are included in the pharmaceutical composition which (optionally together with CRF) induce a cascade for continued production of such molecules. The various components may be provided in combination with one or more carrier molecules which bind one or more of the components, and so act as a depot or reservoir for release of the component. A carrier molecule may also be used in combination with POMC and its related peptides.

According to a further aspect of the present invention, there is provided a method of treatment for a disease selected from multiple sclerosis; rheumatoid arthritis; optic neuritis; motor neurone disease; autoimmune diseases including lupus, psoriasis, eczema, thyroiditis, and polymyositis; axonal or nerve damage; cancers, in particular myelomas, melanomas, and lymphomas; neural disorders, both demyelinating and non-demyelinating; inflammatory conditions; obesity; nerve conduction disorders; and sexual dysfunction, in particular erectile dysfunction; the method comprising administering CRF to a patient in need thereof. Alternatively, the method may comprise administering POMC.

The optimal dosage of the treatment has not yet been determined; however it may be appropriate to administer the treatment in a dosage of between 0.01 and 10 mg/kg to the subject; more preferably between 0.01 and 5 mg/kg, between 0.025 and 2 mg/kg, and most preferably between 0.05 and 1 mg/kg. In preliminary studies, a serum product has been administered to patients with a total protein concentration of 4 mg/ml.

The precise dosage to be administered may be varied depending on such factors as the age, sex and weight of the patient, the method and formulation of administration, as well as the nature and severity of the disorder to be treated. Other factors such as diet, time of administration, condition of the patient, drug combinations, and reaction sensitivity may be taken into account.

An effective treatment regimen may be determined by the clinician responsible for the treatment. One or more administrations may be given, and typically the benefits are observed after a series of at least three, five, or more administrations. Repeated administration may be desirable to maintain the beneficial effects of the composition.

The treatment may be administered by any effective route, preferably by subcutaneous injection, although alternative routes which may be used include intramuscular or intralesional injection, oral, aerosol, parenteral, or topical.

The treatment is preferably administered as a liquid formulation, although other formulations may be used. For example, the treatment may be mixed with suitable pharmaceutically acceptable carriers, and may be formulated as solids (tablets, pills, capsules, granules, etc) in a suitable composition for oral, topical or parenteral administration.

The invention also provides the use of CRF in the preparation of a medicament for the treatment of one or more of the diseases recited above. Also provided is the use of POMC in the preparation of a medicament for the treatment of one or more of the diseases recited above. The CRF or the POMC may be isolated, purified CRF or POMC, although it is preferred that they are administered in combination with the various other components as discussed above. In particular, bioactive carrier proteins and vasopressin may be used.

According to a further aspect of the present invention, there is provided a method of producing CRF, the method comprising the steps of obtaining a blood sample from a goat; separating the serum from the remaining blood components; and purifying the serum by precipitation of solids.

The precipitate may further be resuspended in a physiologically acceptable buffer; for example, PBS buffer. The resuspended precipitate may further be purified by dialysis or diafiltration; for example, with a molecular weight cut-off of 50,000 Da, preferably 40,000 Da, and more preferably 31,000 Da.

The precipitate may undergo further purification to isolate CRF; for example, antibody or other affinity purification. CRF may be bound by antibodies raised against CRF, or by use of CRF-BP.

Separation of the serum may be achieved by centrifugation.

The serum may further be purified by viral filtration; it is preferred that any purification method used does not inactivate or remove any of the bioactive components of the serum, which may include components other than CRF/POMC.

Precipitation may be carried out by ammonium sulphate precipitation, or by caprylic acid purification. Other suitable precipitating agents may be used.

Preferably the goat is an immunized goat. It is believed that immunization of the goat stimulates the production of CRF and vasopressin, such that it is present in the serum in higher levels. The method may also comprise the step of immunizing a goat. Alternatively, the goat may be subject to physiological stress, for example, bleeding.

It is also believed that, in some circumstances, the use of an immunogen may not be necessary, and that useful product may be obtained from a non-immunised animal (that is, one which has not been pre-immunised with a specific immunogen). It is accepted that a normal goat may well have been previously exposed to environmental immunogens, and such goats may be used in preparation of the compositions of the present invention. The present invention is therefore intended also to encompass pharmaceutical compositions comprising serum obtained from a non-immunised goat. The invention also extends to uses of such compositions or such serum in the treatment of, or in the preparation of medicaments for the treatment of, the various diseases or disorders recited above in the section headed “uses of the serum composition”. The invention still further extends to the preparation of POMC and/or CRF from serum obtained from a non-immunised goat.

### BRIEF DESCRIPTION OF THE DRAWINGS

These and other aspects of the present invention will now be described by way of example only with reference to the accompanying drawings, in which:

FIGS. 1 to 4 show mass spectrometry analyses of tryptic digests of serum components;

FIGS. 5 to 7 show mass spectrometry analyses of patient sera before and after treatment with the composition;

FIGS. 8 and 9 show analyses of induction of POMC peptides by treatment with the composition;

FIGS. 10 to 14 show evidence for a switch in inflammatory profile of patients following treatment with the composition;

FIG. 15 shows levels of vasopressin in human serum and the composition;

FIG. 16 shows levels of CRF in human serum and the composition; and

FIG. 17 shows a summary diagram of the proposed elements of the composition and their method of action.

### DETAILED DESCRIPTION OF THE INVENTION

### Preparation of Serum Composition

Approximately 400 cc of blood is taken from a goat under sterile technique. The animal may typically be re-bled in 10 to 14 days, once the volume of blood is replenished. A pre-bleeding regime may be useful to stimulate production of the active components of the serum. The blood is then centrifuged to separate the serum, and the serum filtered to remove large clots and particulate matter. The serum is then treated with supersaturated ammonium sulphate (47% solution at 4° C.) to precipitate antibodies and other material. The resulting solution is centrifuged in a Beckman J6M/E centrifuge at 3500 rpm for 45 minutes, after which the supernatant fluid is removed. The precipitated immunoglobulin and other solid material are resuspended in PBS buffer (phosphate buffered saline) sufficient to redissolve the precipitate.

The solution is then subjected to diafiltration against a PBS buffer with a molecular weight cut-off of 10,000 Daltons. at 4° C. After diafiltration the product is filtered through a 0.2 micron filter into a sterile container and adjusted to a protein concentration of 4 mg/ml. The solution is put into vials to give single doses of 1 ml, and stored at −22° C. prior to use.

### Discussion

The effects of the serum have been previously described, while determination of the active components has not previously been effected.

Analysis of Serum Composition

A sample of the composition was size fractionated on a gel, and a Western blot performed using antibodies to β endorphin. A strong signal was detected, indicating the presence of β endorphin, although the apparent molecular weight was approximately 31 kDa, far larger than the expected size of β endorphin. This suggested that β endorphin was present in the sample as part of a larger peptide; the size being consistent with that of POMC.

We have also carried out mass spectrometry on the composition, and have detected at least two POMC-derived peptides, β endorphin and corticotrophin-related molecules. CRH-BP (corticotropin releasing hormone binding protein) has also been identified.

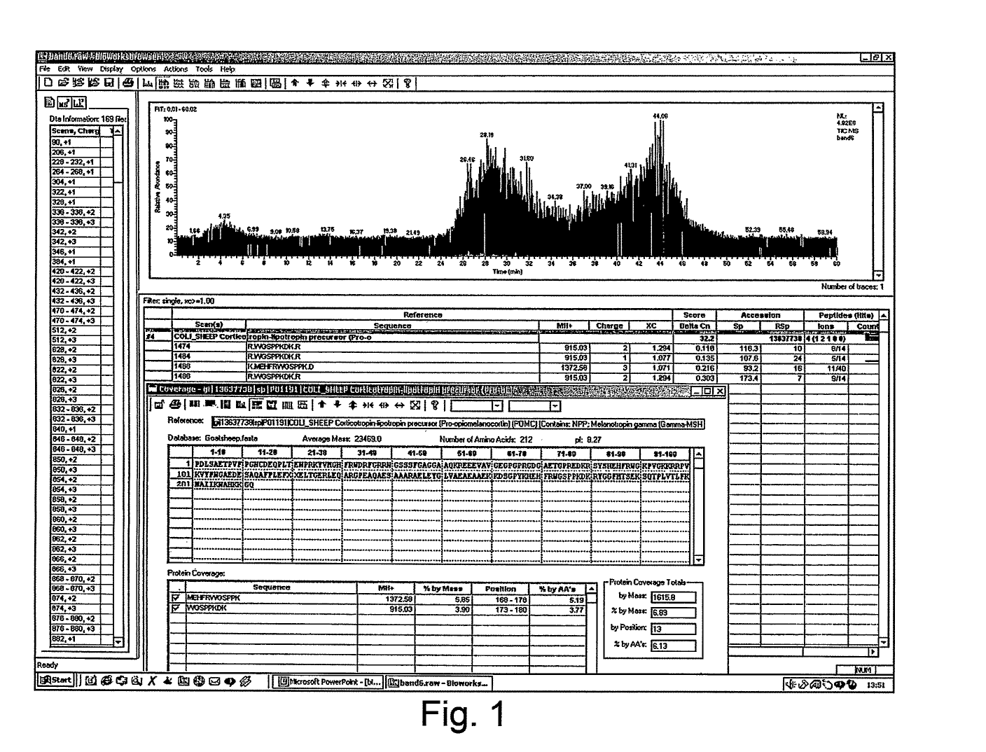

### FIGS. 1 to 4

POMC peptides and CRF-BP have been identified in the product by Thermofinnegan LCQ mass spectrometry. CRF mainly regulates the synthesis and secretion of ACTH in the anterior pituitary. The administration of POMC and/or its component peptides in addition to CRF and CRF-BP is thought to initiate a cascade effect thus enhancing the production of systemic and sustained elevated concentrations of POMC peptides. CRF-BP has the ability to act as a reservoir for CRF.

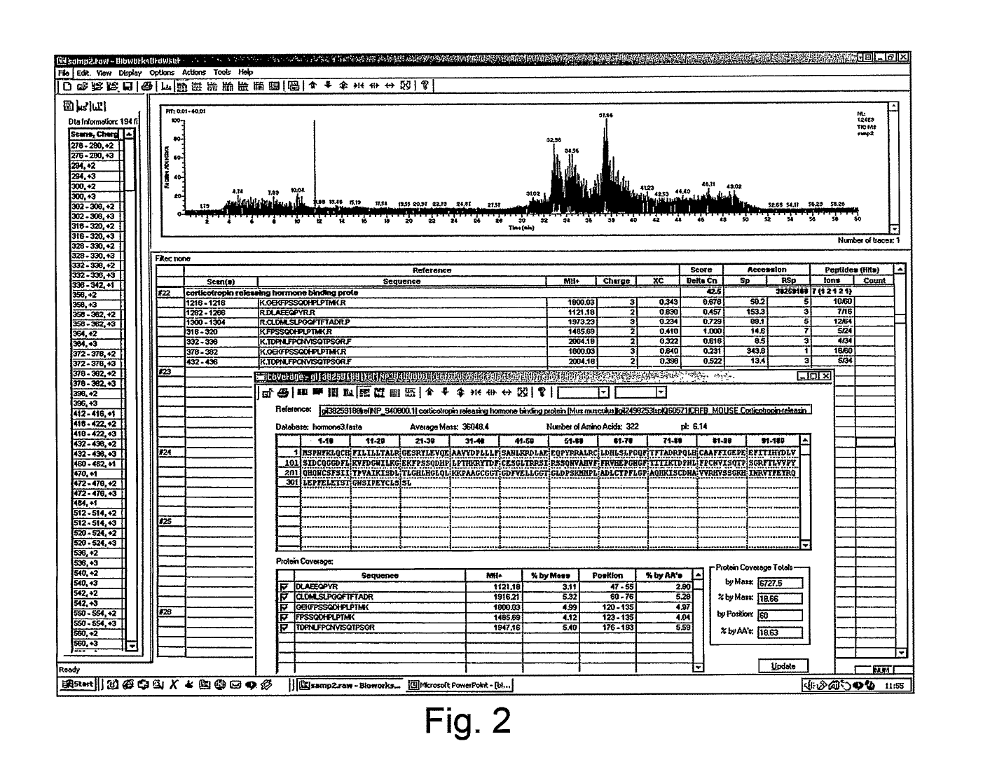

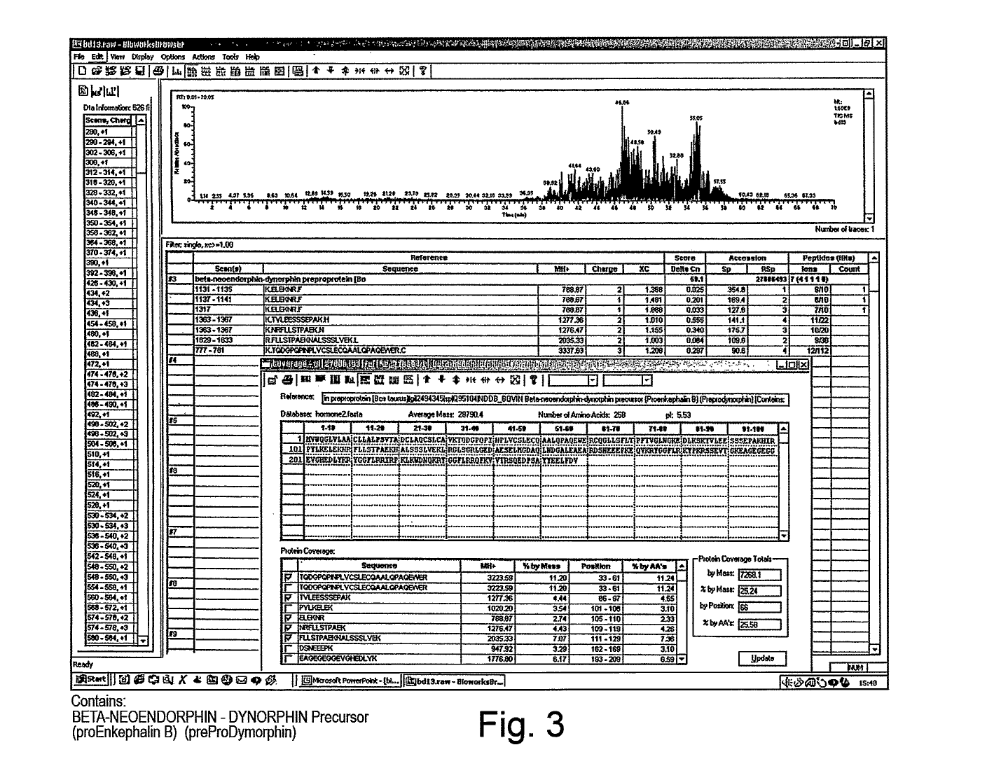

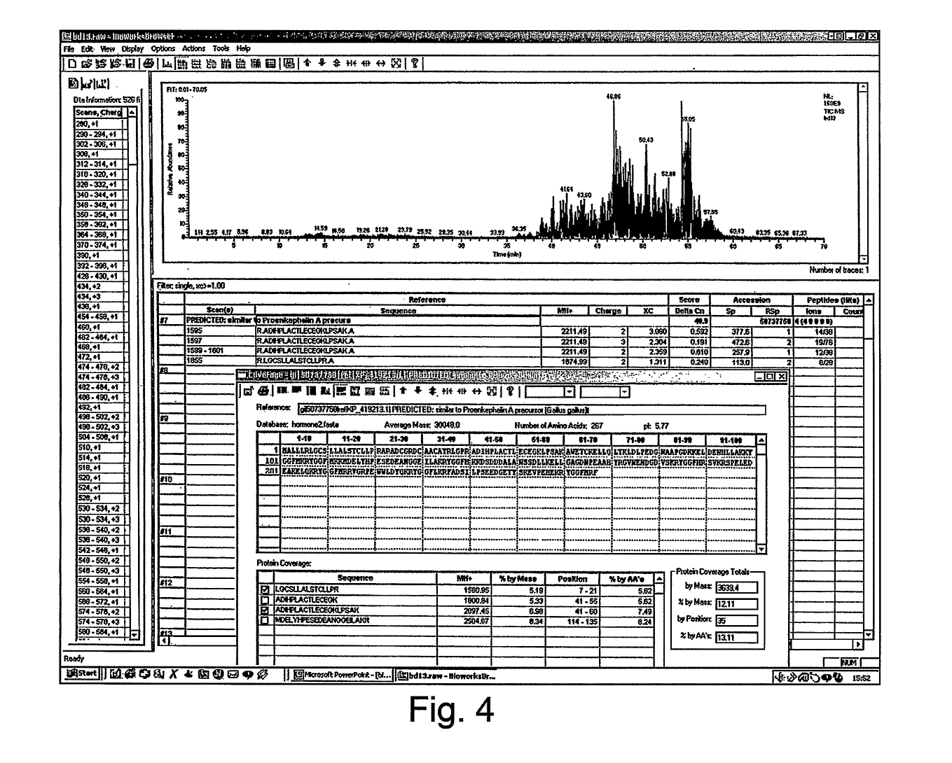

FIGS. 1 to 4 show the hits obtained from mass spectrometry analysis of tryptic digests from the product separated from contaminating proteins by SDS-PAGE. As mentioned above, some of these molecules are inducers and regulators of the POMC cascade. Further investigation using more focused analysis (e.g. peptide fractionation, immunoprecipitation and concentration) will reveal more of the peptides present. FIG. 1 indicates the presence of a POMC-derived corticotropin, FIG. 2 that of CRF-BP, FIG. 3 that of proenkephalin A, and FIG. 4 that of proenkephalin B. The presence of CRF-BP suggests that the product contains some CRF, while POMC and related peptides are also clearly present.

We have also investigated the effects of treatment with the serum composition on patients' own sera. These effects are described below.

### Treatment Induces Protein/Peptide Expression in Patients' Sera

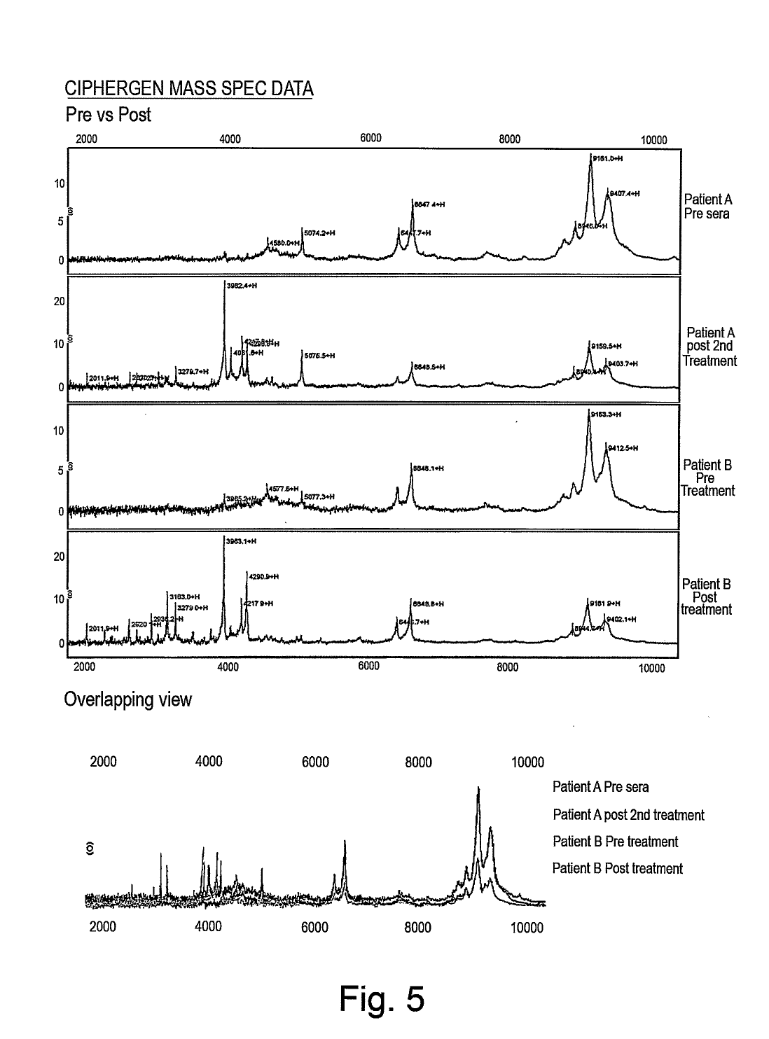

FIG. 5 shows mass spectrometry of patients' sera before and after treatment. The spectra from 2 to 10 kD are compared. This molecular weight range is associated with the bioactive peptides of interest. Clear differences in the peptide expression in the 2 to 6 kD region can be seen by comparing the profiles in the pre and post treatment sera. For ease of comparison an overlapping view of the profiles is also provided.

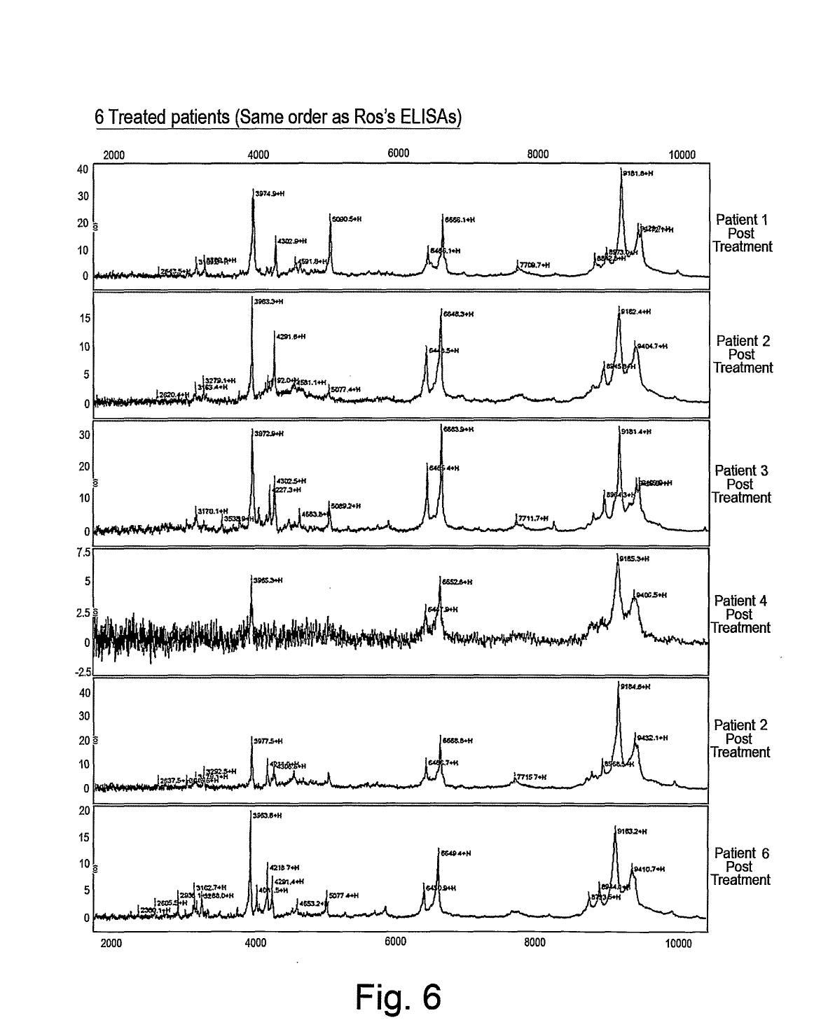

FIG. 6 shows comparative peptide/protein expression in six treated patients. Each patient shows increased levels of induced peptide/protein expression particularly in the 4 kD region.

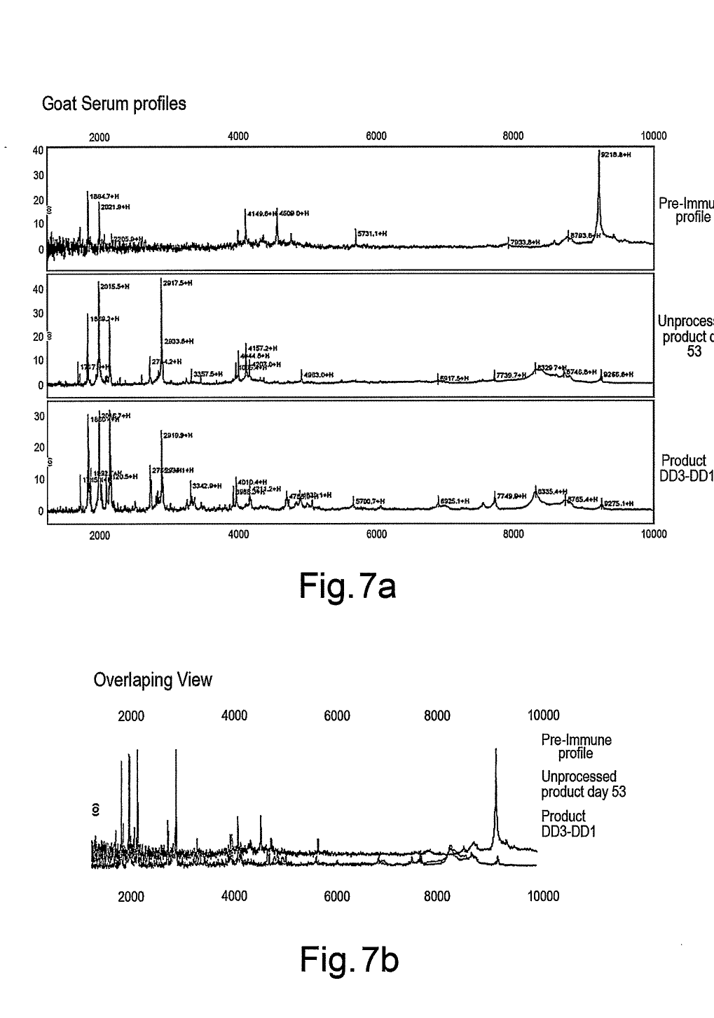

FIG. 7a shows the mass spectrometry profiles of unprocessed goat serum before vaccination (pre-immune profile, top panel), unprocessed serum 53 days post-immunisation, and the processed product. It can be seen that in the lower two panels the profile of the serum is significantly different to that of the pre-immune profile, indicative of the induction of protein expression. The profiles present here represent the active product, and a specific immunisation/bleed protocol has been shown to be useful in the induction of this serum profile. An overlapping view of the profiles is shown (FIG. 7b).

### Evidence for the Induction of POMC Peptides

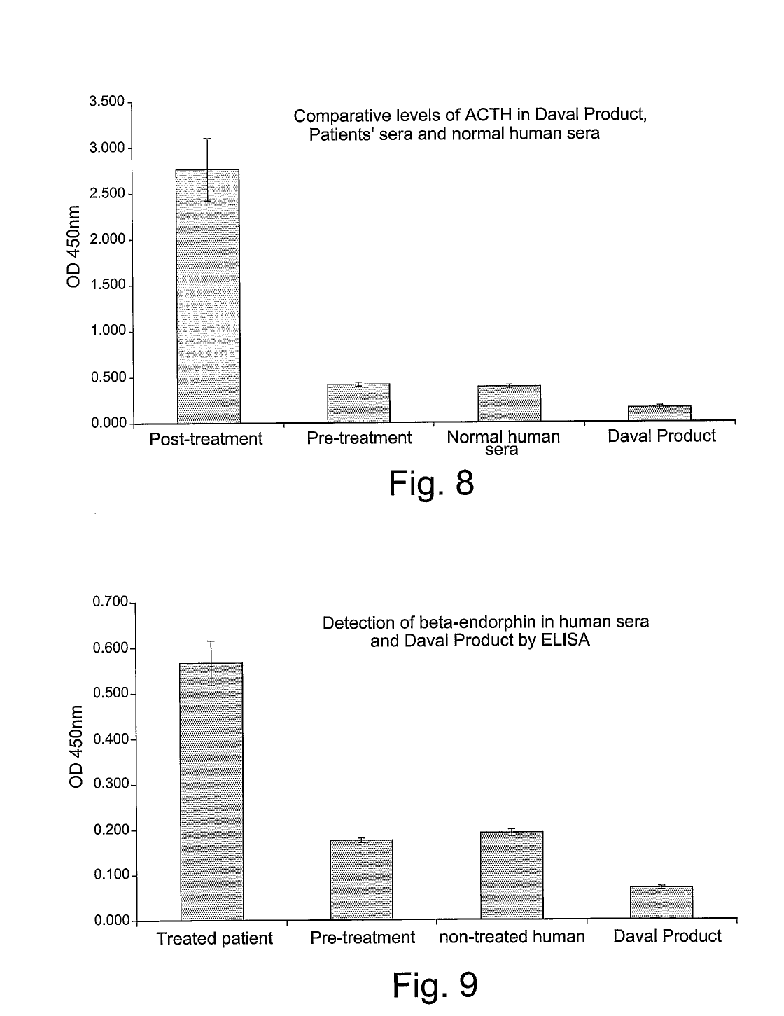

FIG. 8 shows comparative levels of ACTH in the sera of patients before and after receiving treatment. This is also compared with levels of ACTH in serum from healthy volunteers and in the product administered to patients. Sera were diluted 1:100 and quantified by an ELISA of sera compared with the product. Data are the mean of three determinations +/− standard errors. Post treatment n=5; pre treatment n=3; normal human sera n=5. The data show that treatment increases ACTH levels.

FIG. 9 compares levels of p endorphin in the serum of treated patients with that in the sera of the same patients before treatment. This is compared with levels in the sera of healthy volunteers and in the product. Sera were diluted 1:100 and quantified by an ELISA of sera compared with the product. Data are the mean of three determinations +/− standard errors. The data show that treatment increases β endorphin levels.

Evidence for a Switch from a Pro-Inflammatory TH-1 Profile to an Anti-Inflammatory TH-2 Cytokine Profile in Treated Patients

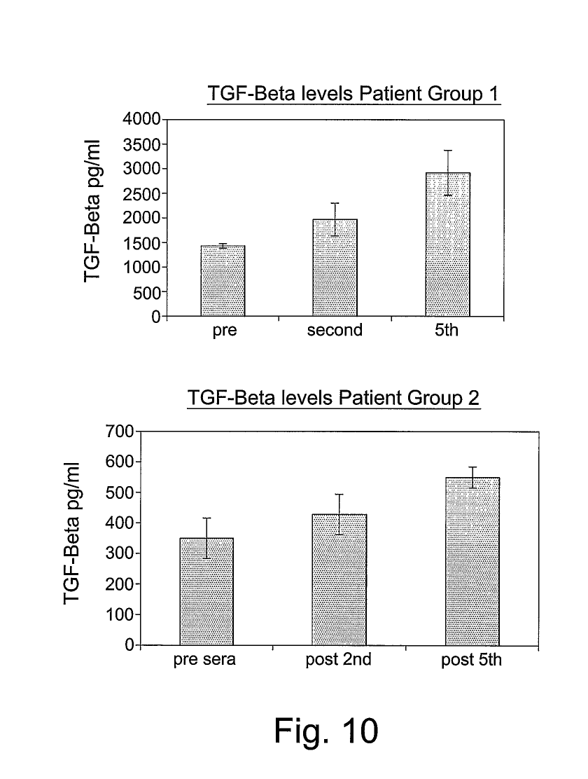

FIG. 10 shows the levels of TGF-β in the serum of two groups of patients before and after treatment. The two groups of patients (n=3 for each group) show differing responses with respect to the concentrations of TGF-β produced, but all patients showed an increase in serum levels in response to treatment (pre sera=patients' serum levels before treatment; post 2^{nd} and post 5^{th}=after the 2^{nd} and 5^{th} administration). The data show that treatment induces increased concentration of the anti-inflammatory cytokine TGF-β.

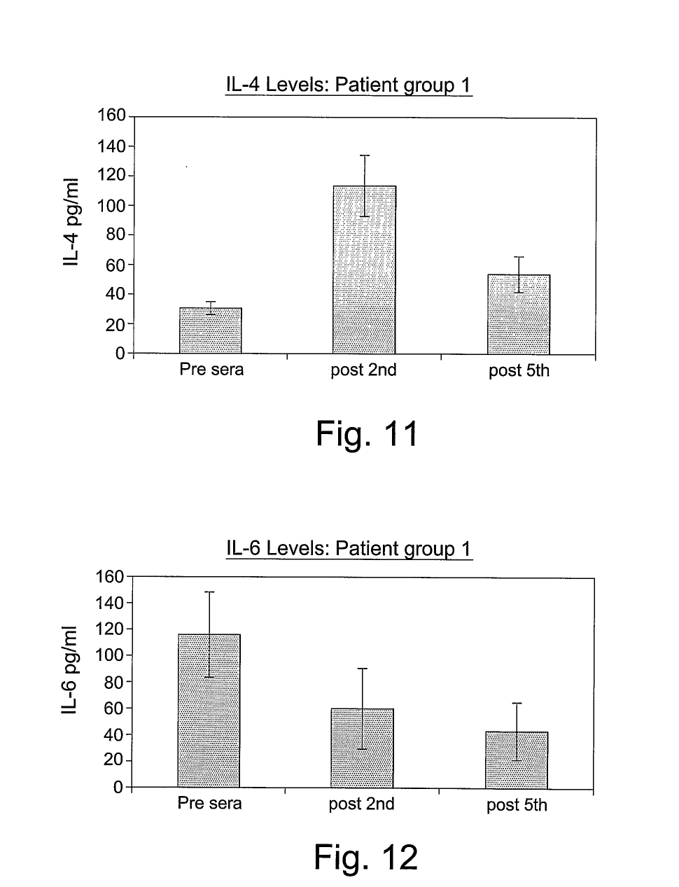

FIG. 11 shows the levels of IL-4 in the serum of one group of patients before (pre-sera) and after treatment. It can be seen that after treatment (post 2^{nd}), the levels of IL-4 are significantly increased in the patients' sera (n=5). However, following the 5^{th} administration, the levels of IL-4 had dropped in all patients, but remained higher than they had been pre-treatment. IL-4 is known to downregulate the production of the pro-inflammatory cytokines from TH-1 cells. It may be that the consistent changes in concentration seen in all patients is consistent with IL-4's role in the TH-1 to TH-2 switch.

FIG. 12 shows the levels of IL-6 in the serum of one group of patients before and after treatment. It can be seen that after treatment (post 2^{nd} and post 5^{th}) the levels of IL-6 are reduced in the patients' sera (n=4).

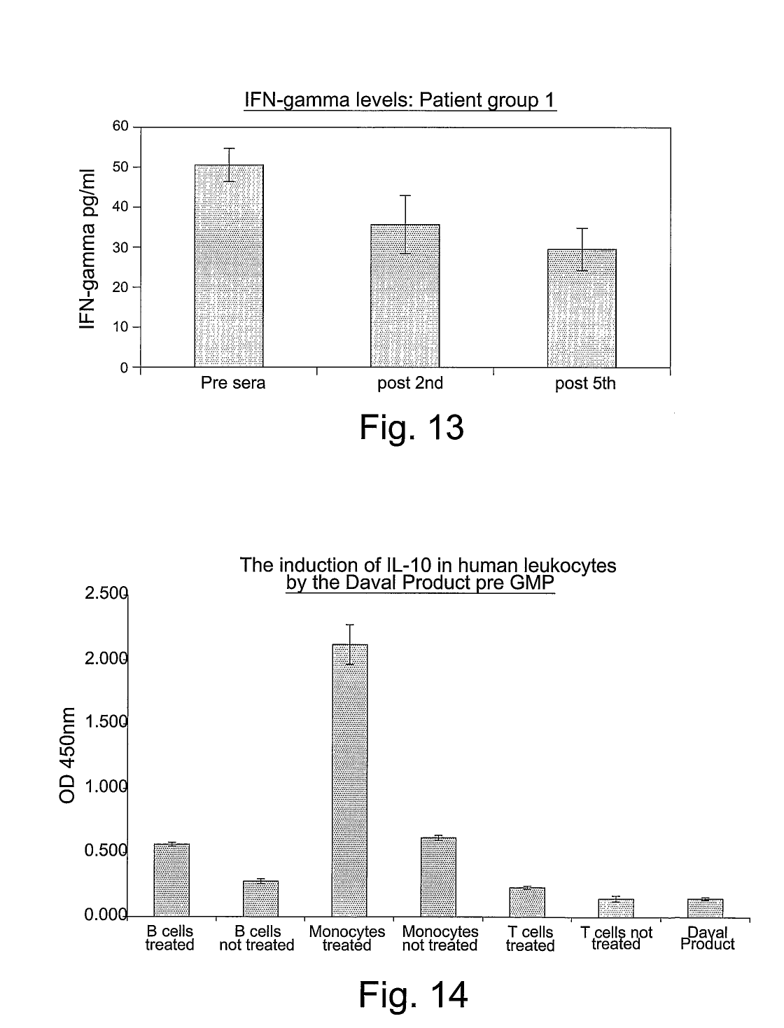

FIG. 13 shows the levels of IFN-γ in the serum of one group of patients before and after treatment. It can be seen that after treatment (post 2^{nd} and post 2^{th}) the levels of IFN-y are reduced in the patients' sera.

FIG. 14 shows that treatment of human peripheral blood cells (PBMCs) induces the production of the anti-inflammatory cytokine IL-10 in the monocyte sub population. T and B lymphocytes and monocytes were separated from PBMCs obtained from human volunteers. All cell types were treated with equivalent doses of product for 16 h, and their supernatants assayed for IL-10 content using ELISA. It can be seen that IL-10 levels produced by the T cell population were unaffected by treatment and that only a small increase in IL-10 was induced in the B cells. However, a significant elevation of IL-10 concentration was induced in the monocytes population by the treatment. All determinations were made in triplicate +/− standard deviations. These data are representative of at least three separate experiments.

### Evidence for Vasopressin and CRF Induction

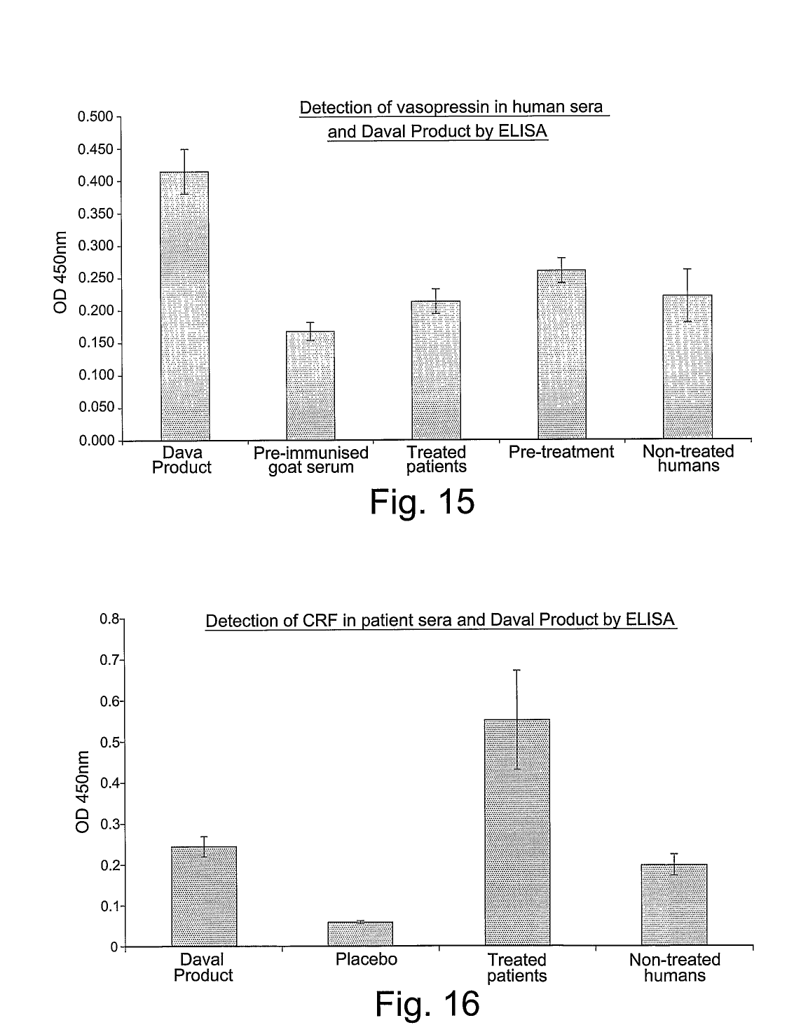

FIG. 15 shows the comparative levels of vasopressin in the product, control patients and patients treated with the product and pre-treatment. The figure shows that there is no significant difference between any of the serum groups, however the product contains significant levels of vasopressin, sufficient to elicit a response in the patients. It is known that vasopressin acts synergistically with CRF to release POMC. All determinations were made in triplicate +/− standard deviations. These data are representative of at least 3 separate experiments. Patients pre-treatment n=3; treated patients n=6.

FIG. 16 shows the increased presence of CRF in the product compared with the placebo and the increase in the treated patients compared with the non-treated individuals; the latter is evidence for the induction of CRF in the patients in response to treatment. All determinations were made in triplicate +/− standard deviations. These data are representative of at least 3 separate experiments. Control individuals n=4; treated patients n=13.

### SUMMARY AND CONCLUSIONS

Although preliminary, the evidence to date is therefore consistent with the major active component being CRF acting in concert with other components, which is thought to induce POMC production. There is also evidence that POMC itself and POMC-derived peptides may be used as a treatment. This suggests new pharmaceutical compositions and uses for CRF and POMC, as well as indicating additional disorders which may be treatable using CRF and POMC. We have also provided a convenient method of producing CRF and POMC from goats.

The data so far suggests that the product not only contains CRF, POMC peptides and anti-inflammatory cytokines (IL-10 and TGF-β) but also induces the expression and release of CRF and hence POMC peptides in the patient which then transform the patients' immunological profile from a TH-1 pro-inflammatory profile to a predominantly TH-2 anti-inflammatory profile.

Other observations on the composition effects are consistent with the active component being CRF which leads to POMC production. For example, effects on leukocyte adherence may be attributable to beta endorphin. The serum product increases IL-10 production by human PBMC; alpha MSH affects IL-10 production. Effects on nerve conduction and neuroprotective effects may be ascribed to ACTH and vasopressin; effects on appetite may be due to alpha MSH. The product itself also contains significant levels of IL-10 and TGF-β (data not shown).

Alpha MSH has potent anti-inflammatory effects in all major forms of inflammation and it antagonises the effects of pro-inflammatory cytokines such as TNFα and IL1-β. Cross talk exists between the cytokine systems and the POMC system which has been observed in patients treated with the composition to result in the reduction of pro-inflammatory cytokines and the establishment (retained over the course of treatment) of a TH-2 anti-inflammatory cytokine profile including elevated levels of IL-10 and TGF-β. We have also identified increased levels of IL1-β in the serum product.

The serum product has previously been shown to be very sensitive to proteolytic degradation; this is consistent with the theory that the POMC is proteolysed to give individual hormones on administration, but that further degradation destroys activity. In particular, alpha MSH is believed to have significantly reduced activity if a terminal tripeptide sequence is removed; again, this is consistent with the active component including POMC. The product itself is unstable by nature as its active components are short-lived, but exhibit powerful effects.

We have also conducted experiments which suggest that the serum modulates nitric oxide production by leukocytes; this is consistent with effects of beta endorphin. We also believe that the serum inhibits PHA-induced PBMC proliferation, suggesting an explanation for the serum's immunomodulatory effects. We have also seen a reduced response of PBMCs in the presence of the product to LPS-induced stimulation and mixed lymphocyte reactions (data not shown).

The product may also induce tyrosine phosphorylation in human brain microglial cells, and has been shown by Western blotting to modulate the NFκB pathway (data not shown). NFκB is known to regulate the transcription of genes involved in the regulation of pro-inflammatory cytokines, hence the inhibition of NFκB would act to reduce the pro-inflammatory cytokine response in autoimmune disease and reduce inflammatory responses. Further experiments to investigate this are underway.

Receptors (MCR3 and MCR4) for some POMC peptides are found in the retinal ganglion cells that form the optic nerve and may be stimulated by POMC peptides produced after treatment. This may account for some of the rapid improvements in vision experienced by MS patients with optic neuritis which have previously been described. It is known that ACTH triggers the corticosteroid pathway which can exert effects in as little as 20 to 30 minutes. Preliminary data suggests that the concentrations of the peptides in the product may be insufficient to elicit therapeutic responses in patients after dilution in the blood volume of the patient. However, the product could act locally (as it is injected in a subcutaneous bolus) to induce a biochemical cascade which triggers the synthesis and release of the bioactive peptides in the treated patients. It is now known that any medical treatments that interfere with the product, for example by competing for receptors or blocking molecules in the HPA should be avoided.

In support of this hypothesis mass spectrometry of the product has identified additional molecules some of which are involved in the induction and regulation of the corticotropin system; namely CRH binding protein and leu-enkephalin, corticotropin-lipotropin precursor and pro-enkephalin A precursors (see FIGS. 1 to 4). In addition, and perhaps more importantly, we have discovered that two of the major POMC peptides are upregulated significantly in treated patients' sera compared with levels before treatment, and also compared with levels from healthy control volunteers. This finding, together with immunological data, suggests that the treatment induces the expression and release of POMC peptides in the patient, which then transforms the patients' immunological profile from a TH-1 pro-inflammatory profile to a TH-2 anti-inflammatory profile. The further elucidation of the cascade mechanism in the patients is currently under investigation.

It should be noted that although the product is anti-inflammatory in nature it does not completely inhibit the inflammatory response. Our data suggest that the product induces a shift from the unfavourable TH-1 cytokine profile seen in auto-immune diseases to a more favourable balanced cytokine level. This may appear initially after treatment as a rapid anti-inflammatory TH-2 shift as the TH-1 network is turned off. Later on after treatment the TH-1 network operates albeit at a lower level.

The reported effects of the serum product on tumours leads us to consider the possibility of anti-angiogenic effects of the serum. In this regard, the proteins thrombospondin-1 (TSP-1) and platelet factor 4 (PF-4) have been identified in the product by mass spectrometry of tryptic digests from SDS PAGE gels. Computer database searches using Biowork Browser for peptide identification yielded strong matches across several species including Homo sapiens. Although precise quantification of the TSP-1 and PF-4 protein content of the product has not yet been established, the visible nature of the protein bands on SDS PAGE gels indicates that the proteins are present in biologically significant (upper nanogram) quantities.

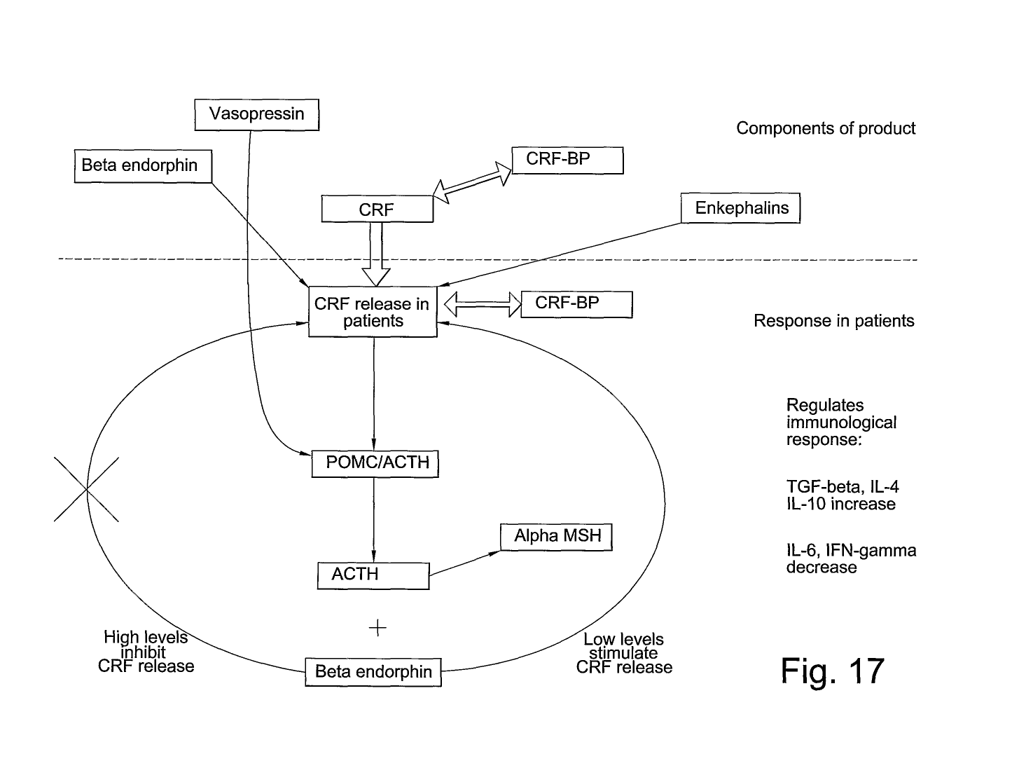

A summary of the hypothesised components of the product, and the method of action, is shown in FIG. 17. The product is thought to contain CRF, with some levels of CRF-BP, beta endorphin, vasopressin, and enkephalins. CRF induces production of further CRF in the patient, as do beta endorphin and the enkephalins. Endogenous CRF causes production of POMC, which gives rise to among others ACTH, alpha MSH, and beta endorphin. This last product acts in a feedback loop, with low levels stimulating further CRF release, while high levels inhibit CRF release. This whole CRF/POMC cascade is thought to induce an immunological switch in the patient, which could explain the surprising beneficial effects seen in a variety of conditions.

1. A pharmaceutical composition comprising a corticotropin releasing factor (CRF) peptide.

2. The composition of claim 1, comprising goat CRF.

3. The composition of claim 1 or 2, further comprising one or more of vasopressin, beta endorphin, and an enkephalin.

4. The composition of any preceding claim, comprising CRF binding protein.

5. The composition of any preceding claim further comprising a POMC peptide.

6. The composition of any preceding claim further comprising one or more of α, β, and γ MSH; ACTH; β and γ LPH; met-enkephalin, leu-enkephalin and beta endorphin.

7. A method of stimulating POMC production in a patient, comprising administering exogenous POMC to the patient.

8. The method of claim 7, further comprising administering exogenous CRF to the patient.

9. The method of claim 7 or claim 8, further comprising administering exogenous vasopressin to the patient.

10. A pharmaceutical composition comprising a POMC peptide.

11. Use of an isolated CRF peptide in the preparation of a medicament.

12. Use of an isolated POMC peptide in the preparation of a medicament.

13. A method of treatment for a disease selected from multiple sclerosis; rheumatoid arthritis; optic neuritis; motor neurone disease; autoimmune diseases including lupus, psoriasis, eczema, thyroiditis, and polymyositis; axonal or nerve damage; cancers, in particular myelomas, melanomas, and lymphomas; neural disorders, both demyelinating and non-demyelinating; inflammatory conditions; obesity; nerve conduction disorders; and sexual dysfunction, in particular erectile dysfunction; the method comprising administering CRF to a patient in need thereof.

14. A method of treatment for a disease selected from multiple sclerosis; rheumatoid arthritis; optic neuritis; motor neurone disease; autoimmune diseases including lupus, psoriasis, eczema, thyroiditis, and polymyositis; axonal or nerve damage; cancers, in particular myelomas, melanomas, and lymphomas; neural disorders, both demyelinating and non-demyelinating; inflammatory conditions; obesity; nerve conduction disorders; and sexual dysfunction, in particular erectile dysfunction; the method comprising administering POMC to a patient in need thereof.

15. A method of producing CRF, the method comprising the steps of obtaining a blood sample from a goat; separating the serum from the remaining blood components; and purifying the serum by precipitation of solids.

16. A method for curative, ameliorative, or prophylactic treatment of a disease selected from:
rheumatoid arthritis; optic neuritis; motor neurone disease; axonal or nerve damage;
cancers, in particular myelomas, melanomas, and lymphomas;
non-demyelinating neural disorders including cerebrovascular ischaemic disease, Alzheimer's disease, Huntingdon's chorea, mixed connective tissue diseases, scleroderma, anaphylaxis, septic shock, carditis and endocarditis, wound healing, contact dermatitis, occupational lung diseases, glomerulonephritis, transplant rejection, temporal arteritis, vasculitic diseases, hepatitis, burns, multiple system atrophy, epilepsy, muscular dystrophy, schizophrenia, bipolar disorder, depression, channelopathies, myaesthenia gravis, pain due to malignant neoplasia, chronic fatigue syndrome, fibromyositis, irritable bowel syndrome, work related upper limb disorder, cluster headache, migraine, and chronic daily headache;
demyelinating disorders including infections of the nervous system, nerve entrapment and focal injury, traumatic spinal cord injury, brachial plexopathy (idiopathic and traumatic, brachial neuritis, parsonage turner syndrome, neuralgic amyotrophy); radiculopathy; channelopathies; and tic douloureux;
autoimmune diseases including lupus, psoriasis, eczema, thyroiditis, and polymyositis;
inflammatory conditions;
hereditary motor and sensor neuropathy of all types; Charcot-Marie-Tooth disease (CMT) types CMT1A, CMT1B, CMT2, CMT3 (Dejerine Sottas disease), CMT4 (Types A, B C and D), X-linked Charcot-Marie-Tooth disease (CMTX); Hereditary Neuropathy with liability to pressure palsies (HNPP)—also called Tomaculous neuropathy; Hereditary Motor and Sensory Neuropathy with Deafness—Lom (HMSNL); Proximal Hereditary Motor and Sensory Neuropathy I Neuronopathy (HMSNP); Hereditary Neuralgic Amyotrophy; Hereditary Sensory and Autonomic Neuropathies (HSAN1, HSAN2, HSAN3 (also called Riley-Day syndrome or familial dysautonomia), HSAN4, HSAN5); Familial Amyloid polyneuropathies (Type I, Type II, Type III, Type IV); Metachromatic Leukodystrophy; Krabbe's Disease; Fabry's Disease; Adrenoleukodystrophy; Refsum's disease (HMSN IV); Tangier Disease; Friedreich's ataxia; Spinal cerebellar ataxia (SCA) all types—SCA1, SCA2, SCA3, SCA4, SCA5, SCA6, SCA7, SCA8, SCA10, SCA11, SCA12, SCA13, SCA14, SCA16; Spinocerebellar Ataxia;
Cockayne's syndrome; and Giant axonal neuropathy;
chronic inflammatory demyelinating polyneuropathy (CIDP), and Guillain-Barre syndrome;
canine atopic dermatitis, canine oral melanoma, and equine pulmonary disorders;
comprising administering CRF to a patient.

17. A method for curative, ameliorative, or prophylactic treatment of a disease selected from:
rheumatoid arthritis; optic neuritis; motor neurone disease; axonal or nerve damage;
cancers, in particular myelomas, melanomas, and lymphomas;
non-demyelinating neural disorders including cerebrovascular ischaemic disease, Alzheimer's disease, Huntingdon's chorea, mixed connective tissue diseases, scieroderma, anaphylaxis, septic shock, carditis and endocarditis, wound healing, contact dermatitis, occupational lung diseases, glomerulonephritis, transplant rejection, temporal arteritis, vasculitic diseases, hepatitis, burns, multiple system atrophy, epilepsy, muscular dystrophy, schizophrenia, bipolar disorder, depression, channelopathies, myaesthenia gravis, pain due to malignant neoplasia, chronic fatigue syndrome, fibromyositis, irritable bowel syndrome, work related upper limb disorder, cluster headache, migraine, and chronic daily headache;
demyelinating disorders including infections of the nervous system, nerve entrapment and focal injury, traumatic spinal cord injury, brachial plexopathy (idiopathic and traumatic, brachial neuritis, parsonage turner syndrome, neuralgic amyotrophy); radiculopathy; channelopathies; and tic douloureux;
autoimmune diseases including lupus, psoriasis, eczema, thyroiditis, and polymyositis;
inflammatory conditions;
hereditary motor and sensor neuropathy of all types; Charcot-Marie-Tooth disease (CMT) types CMT1A, CMT1B, CMT2, CMT3 (Dejerine Sottas disease), CMT4 (Types A, B C and D), X-linked Charcot-Marie-Tooth disease (CMTX); Hereditary Neuropathy with liability to pressure palsies (HNPP)—also called Tomaculous neuropathy; Hereditary Motor and Sensory Neuropathy with Deafness—Lom (HMSNL); Proximal Hereditary Motor and Sensory Neuropathy/Neuronopathy (HMSNP); Hereditary Neuralgic Amyotrophy; Hereditary Sensory and Autonomic Neuropathies (HSAN1, HSAN2, HSAN3 (also called Riley-Day syndrome or familial dysautonomia), HSAN4, HSAN5); Familial Amyloid polyneuropathies (Type I, Type II, Type III, Type IV); Metachromatic Leukodystrophy; Krabbe's Disease; Fabry's Disease; Adrenoleukodystrophy; Refsum's disease (HMSN IV); Tangier Disease; Friedreich's ataxia; Spinal cerebellar ataxia (SCA) all types—SCA1, SCA2, SCA3, SCA4, SCA5, SCA6, SCA7, SCA8, SCA10, SCA11, SCA12, SCA13, SCA14, SCA16; Spinocerebellar Ataxia; Cockayne's syndrome; and Giant axonal neuropathy;
chronic inflammatory demyelinating polyneuropathy (CIDP), and Guillain-Barre syndrome;
canine atopic dermatitis, canine oral melanoma, and equine pulmonary disorders;
comprising administering POMC to a patient.
